(19) United States
(12) Patent Application Publication
(10) Pub. No.: US 2006/0167099 A1
(43) Pub. Date: Jul. 27, 2006

(54) Use of compositions that increase glutamate receptor activity in treatment of brain injury

(76) Inventors: Anat Biegon, Port Jefferson, NY (US); Esther Shohami, Mevaseret-Zion (IL)

Correspondence Address: Margaret C. Bogosian Esq.;Brookhaven Science Associates, LLC, Building 475D, P.O. Box 5000, Upton, NY, 11973-5000, US

(21) Appl. No.: 10/984,556

(22) Filed: Nov. 9, 2004

(51) Int. Cl. A61K 31/198; A61K 31/195

(52) U.S. Cl. 514/561; 514/567

(57) ABSTRACT

The present invention provides a method for treating a brain injury. This method comprises administering to a mammal afflicted with a brain injury a pharmaceutical composition therapeutically effective to increase glutamate receptor activity in the brain of said mammal. The pharmaceutical composition is to be administered after an acute postinjury phase of said affliction, a time when the level of NMDA receptor activity in the brain is below normal. The pharmaceutical composition may be administered subsequent to an initial treatment with an NMDA antagonist, the NMDA antagonist being administered during the acute postinjury phase of said affliction, a time when the level of NMDA receptor activity is above normal.

### GOVERNMENT SUPPORT

This invention was made in part with government support under Department of Energy Grant KP140102 to A. Biegon. The government has certain rights in the invention.

### BACKGROUND OF THE INVENTION

Head trauma is a leading cause of mortality and morbidity among young people in the western world (Waxweiler et al., J. Neurotrauma 12, 509-516 (1995)). Traumatic and ischemic brain injury triggers a large, transient increase in excitatory amino acid transmitter efflux in the brain of experimental animals and human subjects (Benveniste et al., J. Neurochem. 43, 1369-1374 (1984); Nilsson et al., J. Cereb. Blood Flow Metab. 10, 631-637 (1990); Schuhmann et al., J. Neurotrauma 20, 725-743 (2003); Bullock et al., Ann. N.Y. Acad. Sci. 765, 290-297 (1995); Davalos et al., Stroke 28, 708-710 (1997)). Glutamate activation of the N-methyl-D-aspartate (NMDA) receptor (NMDAR), which is a ligand-gated ion (calcium and sodium) channel, results in channel opening and ion influx into the cell. It has been suggested that this process mediates delayed excitotoxic neuronal death after brain ischemia and trauma (Faden et al., Science 244, 798-800 (1989); Choi et al., Annu. Rev. Neurosci. 13, 171-182 (1990)), although the concept is not universally accepted (Obrenovitch & Urenjak, J. Neurotrauma 14, 677-698 (1997); Obrenovitch et al., Int. J. Dev. Neurosci. 18, 281-287 (2000)). Support for the involvement of NMDAR activation in neuronal death after brain injury has come from numerous studies showing that NMDAR antagonists reduce cell death and improve outcome in animal models of traumatic brain injury (TBI) and stroke. NMDAR antagonists appear to be most efficacious when given before or immediately after the insult and lose efficacy if administered at least 30-60 min postinjury (Rod & Auer, Can. J. Neurol. Sci. 16, 340-344 (1989); Shapira et al., J. Neurotrauma 7, 131-136 (1990); Chen et al., Ann. Neurol. 30, 62-70 (1991); Di & Bullock, J. Neurosurgery 85, 655-661 (1996); Kroppenstedt et al., J. Neurotrauma 15,.191-197 (1998)).

Studies of energy metabolism after human and rodent TBI demonstrate large dynamic changes occurring within the first hour after TBI, such that a hypermetabolic state lasting only 30 min (in rats) to a few hours (in humans) is followed by a profound depression lasting 5 and 30 or more days in rats and humans, respectively (Yoshino et al., Brain Res. 561, 106-119 (1991); Moore et al., J. Cereb. Blood Flow Metab. 20, 1492-1501 (2000); Bergsneider et al., J. Head Trauma Rehabil. 16,135-148 (2001)). Epileptic activity and reductions in ATP content are also restricted to the acute phase after brain injury in rats (Nilsson et al., Brain Res. 637, 227-232 (1984); Mautes et al., J. Mol. Neurosci. 16, 33-39 (2001)). Stimulation of NMDAR is thought to be crucial for memory formation in the mammalian brain (Izquierdo, I., Trends Pharmacol. Sci. 12, 128-129 (1991)), and cognitive impairments are extremely common and often long lasting after TBI (Levin, H. S., J. Clin. Exp. Neuropsychol. 12, 129-153 (1990); Hoofien et al., Brain Injury 15, 189-209 (2001)), suggesting a functional deficit rather than excessive stimulation of NMDAR in the chronic stage after TBI.

### DESCRIPTION OF RELATED PRIOR ART

The increase in excitatory amino acid neurotransmitter release following traumatic and ischemic brain injuries has been described as relatively short-lived in several different animal models (Benveniste et al., J. Neurochem. 43, 1369-1374 (1984); Nilsson et al., J. Cereb. Blood Flow Metab. 10, 631-637 (1990); Schuhmann et al., J. Neurotrauma 20, 725-743 (2003); Obrenovitch & Urenjak, J. Neurotrauma 14, 677-698 (1997); Obrenovitch et al., Int. J. Dev. Neurosci. 18, 281-287 (2000)), whereas the duration in humans is controversial. Some studies report elevations of extracellular glutamate for days after injury, and-others report normalization within 6 h (Bullock et al., Ann. N.Y. Acad. Sci. 765, 290-297 (1995); Davalos et al., Stroke 28, 708-710 (1997)). However, for released glutamate to have a calcium influx-dependent deleterious effect on neuronal survival, it needs to activate its receptors. Prior to the present invention, the extent and duration of NMDAR activation in animal or human posttraumatic or postischemic brains had not been reported.

For the last couple of decades, it was believed that excess stimulation of brain receptors for the excitatory neurotransmitter glutamate was a major cause of delayed neuronal death after head injury. Microdialysis studies of extracellular glutamate in human TBI and stroke patients suggested that the increase in glutamate in humans is more sustained [6 h to several days (Faden et al., Science 244, 798-800 (1989); Choi et al., Annu. Rev. Neurosci. 13,171-182 (1990))] than in rodents, where it only lasts minutes (Benveniste et al., J. Neurochem. 43, 1369-1374 (1984); Nilsson et al., J. Cereb. Blood Flow Metab. 10, 631-637 (1990); Schuhmann et al., J. Neurotrauma 20, 725-743 (2003); Bullock et al., Ann. N.Y. Acad. Sci. 765, 290-297 (1995); Davalos et al., Stroke 28, 708-710 (1997)). This result may have contributed to a decision to administer NMDAR antagonists in human clinical trials of head injury for several days rather than once after severe nonpenetrating injury. That human TBI and stroke patients would require prolonged (3-7 days) NMDAR blockade to achieve therapeutic efficacy was the prevailing dogma, even despite the fact that animal models clearly showed loss of efficacy within 1 h postinjury (Rod & Auer, Can. J. Neurol. Sci. 16, 340-344 (1989); Shapira et al., J. Neurotrauma 7, 131-136 (1990); Chen et al., Ann. Neurol. 30, 62-70 (1991); Di & Bullock, J. Neurosurgery 85, 655-661 (1996); Kroppenstedt et al., J. Neurotrauma 15, 191-197 (1998)). In clinical trials, however, glutamate N-methyl-D-aspartate (NMDA) receptor blockers failed to show efficacy for treating severely head injured patients. Some of these trials were even halted prematurely because of increases in mortality and morbidity in the drug arm of the stroke trials (Fisher, M., Eur. Neurol. 40, 6566 (1998); Maas et al., Neurosurgery 44, 1286-1298 (1999); Narayan et al., J. Neurotrauma 19, 503-557 (2002)), suggesting that prolonged blockade of NMDAR may actually be harmful in the posttraumatic or postischemic patient. Currently there exist no alternative treatment options for the clinician. Accordingly, there exists a need for alternative therapeutic methods that are effective in treating brain injuries.

### SUMMARY OF THE INVENTION

Provided herein is a method for treating a brain injury. This method comprises administering to a mammal afflicted with a brain injury a pharmaceutical composition therapeutically effective to increase glutamate receptor activity in the brain of said mammal. The pharmaceutical composition is administered only after an acute postinjury phase of said affliction, when NMDA receptor activity in the injured brain is below normal. Wherein the mammal is a human, the pharmaceutical composition is administered at a timepoint of at least about 6 hours postinjury. The pharmaceutical composition may be administered intermittently or continuously resulting in a duration of at least 48 hours treatment and may be administered locally to the brain or systemically.

In embodiments of the present invention, the pharmaceutical composition effective to increase glutamate receptor activity may affect glutamate receptor activity either directly or indirectly. The pharmaceutical composition may comprise a glutamate receptor agonist, and the glutamate receptor agonist may be selected from the group consisting of an indirect glutamate receptor agonist, a direct glutamate receptor agonist, and a partial glutamate receptor agonist. The glutamate receptor agonist may further be selected from the group consisting of an NMDA receptor agonist, a Kainate receptor agonist, and an AMPA receptor agonist. Alternatively, the pharmaceutical composition administered may increase a release of glutamate from cells in the injured brain, inhibit the uptake of glutamate by cells in the injured brain, or increase expression of a glutamate receptor in the injured brain. The pharmaceutical composition may comprise a positive modulator of glutamate receptor activity or a glutamate transport inhibitor.

A brain injury to be treated in conjunction with the methods of the present invention may include any brain injury characterized by a decrease in glutamate receptor activity in the injured brain. The brain injury may be caused by an event selected from the group consisting of trauma, ischemia, irradiation, meningitis, surgery, and encephalitis. The present invention may be utilized to treat a brain injury wherein the brain injury is caused by ischemia and the ischemia is caused by a stroke.

The methods of the present invention may comprise the sole therapeutic regimen for treating a brain injury, or the methods may be used in conjunction with other therapies. In either case, more than one pharmaceutical composition therapeutically effective to increase glutamate receptor activity may be administered during treatment of an injury to the brain. A method for treating a brain injury may further comprise first administering to a mammal in need of such treatment a pharmaceutical composition comprising a glutamate receptor antagonist, wherein the composition is administered prior to or during an acute postinjury phase of said affliction; and thereafter administering to the mammal a pharmaceutical composition therapeutically effective to increase glutamate receptor activity in the brain of said mammal, wherein the pharmaceutical composition is only administered after the acute postinjury phase of said affliction. All aforementioned methods of the present invention relating to administration of a pharmaceutical composition therapeutically effective to increase glutamate receptor activity in the brain of said mammal may be used in conjunction with this combination therapy.

### BRIEF DESCRIPTION OF THE DRAWINGS

FIG. 1 illustrates an activation of NMDAR through systemic agonist administration which improves neurological recovery after CHI (closed head injury). Mice subjected to CHI were treated with NMDA, NMDA plus MK801, or vehicle and difference neurological severity score (dNSS) calculated as described in the Exemplification. dNSS was significantly higher in the NMDA-treated animals (top curve, open symbols) compared to vehicle-treated CHI mice (middle curve) at 7 days (P equals 0.05) and 14 days (P equals 0.016) by the Mann-Whitney nonparametric t test. Administration of MK801 (1 mg/kg) in addition to NMDA obliterated the beneficial effect of the agonist (bottom curve). NMDA plus MK801 animals had significantly lower dNSS values (P less than 0.001) at 7 (P equals 0.001) and 14 (P less than 0.0001) days compared to NMDA alone. Recovery was also significantly worse when compared to vehicle at 7 (P equals 0.017) and 14 (P equals 0.05) days.

FIG. 2 shows that activation of NMDAR through systemic agonist administration improves cognitive performance in the object recognition test after CHI. Results are means and standard errors of eight to nine animals per treatment group. Mice were subjected to the object recognition test 14 days after CHI. Injured, vehicle-treated animals (filled bars) lost the ability to recognize the new object, whereas NMDA treated CHI animals (gray bars) spent a significantly higher percentage of their exploration time-near the novel object (*, P less than 0.0001; Student's t test) and were indistinguishable from intact untreated animals (open bars). NMDA at the-dose administered in this study had no effect on the performance of intact animals (hatched bars).

### DETAILED DESCRIPTION OF THE INVENTION

The hypothesis that NMDAR may undergo large and dynamic changes in availability and responsiveness after TBI was tested in a mouse model of closed head injury (CHI) (Chen et al., J. Neurotrauma 13, 557-568 (1996); Yatziv et al., J. Cereb. Blood Flow Metab. 22, 971-978 (2002)) produced by a free-falling weight impacting the intact skull, in combination with quantitative autoradiography of the radiolabeled NMDAR noncompetitive antagonist MK801 (Bowery et al., Br. J. Pharmacol. 93, 944-954 (1988); Porter & Greenamyre, Neurosci. Lett. 69, 105-108 (1994)). Because noncompetitive NMDAR antagonists (e.g., TCP and MK801) are thought to be “use-dependent” ligands (i.e., they bind to a site inside the channel made accessible when the receptor is activated by glutamate (Foster & Wong, Br. J. Pharmacol. 91, 403-409 (1987)), an increase in binding of agents of this class served as a functional marker of excessive NMDAR activation in the brain (Wallace et al., J. Neurosurg. 76, 127-133 (1992); Owens et al., Nucl. Med. Biol. 27, 557-564 (2000)).

CHI was indeed determined to increase the density of MK801 binding and hence the activation of NMDAR in the brain. However, the increase was short-lived, in agreement with the reported therapeutic window of NMDAR antagonists in models of brain injury, and region-specific, being most pronounced in the hippocampus, a region subserving memory functions. Regions at the level of impact had decreased, rather than increased, MK801 binding even at 15 min postinjury, probably reflecting an earlier onset and even shorter (less than 15 min) duration of activation at the site of impact. To Applicant's knowledge, this is the first report of acute (less than 1 h) changes in MK801 binding in the posttraumatic brain. These early changes are likely largely reversible, since washing the brain sections in buffer for 60 min at room temperature, a procedure apt to remove endogenously released glutamate, reversed the effect.

At sixty minutes postinjury, a significant bilateral decrease in activated NMDAR was observed both at the level of impact and at more posterior levels. Persistent and progressive decreases were observed within the first 24 h after the injury. These reductions were only partially reversible by washing the tissue sections for 60 min at room temperature, a procedure apt to remove not only glutamate but also endogenous, water-soluble inhibitors of NMDAR channels as described by others (Porter & Greenamyre, Neurosci. Lett. 69, 105-108 (1994); Quirion & Pert, NIDA Res. Monogr. 43, 217-223 (1983), McCoy & Richfield, Brain Res. 710, 103-111 (1996)). The earliest decreases in functional NMDAR described in this study are compatible with a CHI-induced desensitization and early increase in the level or activity of this inhibitory factor, which does not appear to be magnesium (Quirion & Pert, NIDA Res. Monogr. 43, 217-223 (1983)). While not wishing to be bound by theory, this decrease in functional NMDAR may constitute one of the first lines of defense mounted by the brain against the excessive stimulation of NMDAR after injury. The progressive decrease in functional NMDAR between 1 and 24 h postinjury most likely reflects contributions from additional mechanisms, such as reductions in NMDAR density and gene expression, which have been reported to occur within a few hours and last up to several days after different models of inflammatory, ischemic, or traumatic brain injury (Biegon et al., J. Neurochem. 82, 924-934 (2002); Miller et al., Brain Res. 526,103-107 (1990); Friedman et al., Brain Res. Mol. Brain Res. 86,-34-47 (2001); Sihver et al., J. Neurochem. 78, 417-423 (2001)). In accordance with this hypothesis, administration of the NMDAR agonist NMDA to animals with CHI 1 and 2 days after the injury was found to significantly improve general neurological and cognitive function assessed 2 weeks postinjury. Moreover, coadministration of MK801 and NMDA resulted not only in abrogation of the beneficial effects of NMDA, but in prolongation and aggravation of neurological deficits seen in injured, vehicle-treated mice, probably because of blockade of endogenous glutamate as well as the administered agonist. Similarly, early administration of MK801 (Barth et al., Stroke 11, 153-157 (1990)) has been shown to be beneficial for treating brain injury, while a delayed administration of MK801 has been determined to induce long-lasting (7 days) exacerbation of brain injury-related deficits (Rod & Auer, Can. J. Neurol. Sci. 16, 340-344 (1989); Shapira et al., J. Neurotrauma 7, 131-136 (1990); Chen et al., Ann. Neurol. 30, 62-70 (1991); Di & Bullock, J. Neurosurgery 85, 655-661 (1996); Kroppenstedt et al., J. Neurotrauma 15, 191-197 (1998)).

Accordingly, the present invention is based, in part, on the finding that regions of brain tissue undergo dynamic changes in activation of NMDA receptors in response to brain injury. With the use of the stated mouse model of traumatic brain injury, the present invention provides the first evidence that hyperactivation of glutamate NMDA receptors after injury is short-lived (<1 h in mouse) and is followed by a profound and long-lasting (>7 days) loss of function. Furthermore, the present invention provides the first evidence that stimulation of NMDA receptors in the mouse model by NMDA 24 and 48 h postinjury produces a significant attenuation of neurological deficits (blocked by coadministration of MK801) and restores cognitive performance 14 days postinjury. The results presented herein provide the underlying mechanism for the well known but heretofore unexplained short therapeutic window of glutamate antagonists after brain injury and support a pharmacological intervention with a relatively long (>24 h) time window easily attainable for treatment of human accidental head injury. The results shed light on several persistent inconsistencies regarding the role of glutamate in the long-term outcome of brain injury. Excessive activation followed within a relatively short time by desensitization and loss of functional NMDAR may explain the preclinical and clinical experience with NMDAR antagonists and suggests alternative, more effective modes of treatment.

In one embodiment, the present invention provides a method for treating a brain injury. This method comprises administering to a mammal afflicted with a brain injury a pharmaceutical composition therapeutically effective to increase glutamate receptor activity in the brain of said mammal. The term “pharmaceutical composition” as used herein describes any molecule, e.g., protein, nucleic acid, or small molecule, with the capability of increasing glutamate receptor activity in the brain of said mammal. The pharmaceutical composition is administered in an amount and for a period of time, effective to improve motor and cognitive function in the mammal. In this method, the pharmaceutical composition is only administered after an acute postinjury phase of said affliction. The term “acute postinjury phase” of said affliction is intended to include any time following injury, during which time functional NMDA receptor activity is increased to a level above normal. The phrase “following acute postinjury phase of said affliction” is intended to include any time following injury, during which time functional NMDA receptor activity is decreased to a level below normal. Treatment may begin at any time after an acute postinjury phase. Although treatment may begin as early as the onset of the initial hypoactivation of NMDA receptor, it is not an absolute requirement of the present invention.

Because traumatic and ischemic brain injuries have been surmised to share mechanistic similarities in glutamate response to insult, one of skill in the art would recognize that the present invention has general applicability for treating a brain injury resulting from a variety of events. It is an object of the present invention to provide methods for treating a brain injury caused by non-limiting events such as trauma, ischemia, irradiation, meningitis, surgery, and encephalitis. The methods of the present invention may be used to treat a brain injury caused by ischemia, wherein the ischemia is caused by a stroke. The aforementioned methods of the present invention may be used to treat any brain injury characterized by a hypoactivation of NMDAR.

Since-glutamate and glutamate receptors are conserved in mammals, a pharmaceutical composition therapeutically effective to increase glutamate receptor activity may be administered to any mammal in conjunction with the methods of the present invention. In all methods of the present invention, the mammal may be, for example, a mouse or human. The methods of the present invention may be practiced for therapeutic or research purposes.

In the Exemplification section which follows, evidence is provided-showing that hyperactivation of glutamate NMDA receptors after injury lasts less than one hour in a mouse model of closed head injury, after which time a sustained loss of function is observed. The observed time frame for hyperactivation of NMDAR in the mouse is consistent with reports in the art showing an increase in extracellular glutamate within the same time frame (Benveniste et al., J. Neurochem. 43, 1369-1374 (1984); Nilsson et al., J. Cereb. Blood Flow Metab. 10, 631-637 (1990); Schuhmann et al., J. Neurotrauma 20, 725743 (2003); Bullock et al., Ann. N.Y. Acad. Sci. 765, 290-297 (1995); Davalos et al., Stroke 28, 708-710 (1997)). Wherein the mammal is a rodent in methods of the present invention, a pharmaceutical composition therapeutically effective to increase glutamate receptor activity in the brain of said rodent is preferably administered at a timepoint of at least about one hour postinjury. Because an increase in extracellular glutamate in the brain of humans is known to last on the order of 6 h (hours) to several days (Faden et al., Science 244, 798-800 (1989); Choi et al., Annu. Rev. Neurosci. 13, 171-182 (1990)), a pharmaceutical composition therapeutically effective to increase glutamate receptor activity in the brain of a human is preferably administered at a timepoint of at least about 6 hours postinjury.

Provided herein is evidence that two doses of the pharmaceutical composition, administered 1 and 2 days postinjury in conjunction with the methods of the present invention, is effective for significantly improving motor and cognitive function in an afflicted mammal. While a single dose is likely to be beneficial to the injured mammal by preventing at least some of the damage incurred by hyperactivated glutamate receptors, in preferred embodiments the pharmaceutical composition is administered intermittently or continuously resulting in a duration of at least 48 hours treatment. The beneficial effects of NMDA treatment were determined to outlast the treatment period by more than a week in the mouse model, suggesting that the accelerated recovery is not dependent on chronic exposure to a pharmaceutical composition of the present invention. The results also suggest that cognitive and neurological recovery is long lasting and irreversible following cessation of treatment. An afflicted individual would likely benefit from longer treatments as opposed to shorter ones since an decrease in glutamate receptor activation following injury is sustained for a period of at least several days in humans and perhaps longer. While there are no strict restrictions on duration of treatment, it is essential that the treatment continue only as long as endogenous glutamate receptor activity is below normal, or alternatively, as long as treatment maintains glutamate receptor activity at a safe level. Intermittent or continuous treatment of a human with the pharmaceutical composition may continue for more than a week, more than 30 days, or even indefinitely following injury.

In the methods of the present invention, the pharmaceutical composition comprises any compound therapeutically effective to increase glutamate receptor activity in the brain of a mammal afflicted with a brain injury. Increasing glutamate receptor activity would sensitize NMDAR-containing cells to the effects of glutamate already present in the extracellular space. Several classes of compounds with the ability to increase glutamate receptor activity in the brain of a mammal are known in the art. It is an object of the present invention that any compound may be used in the methods of the present invention, which upon administration, achieves said result of increasing glutamate receptor activity. The pharmaceutical composition may comprise, for example, a glutamate receptor agonist. The glutamate receptor agonist may be selected from the group consisting of an indirect (allosteric) glutamate receptor agonist, a direct glutamate receptor agonist, and a partial glutamate receptor agonist. The glutamate receptor agonist may further be selected from the group consisting of an NMDA receptor agonist, a Kainate receptor agonist, and an AMPA receptor agonist. The term “NMDA agonist” in the context of the present invention refers to a compound which binds to the NMDA receptor with an affinity that is at least 10 times, preferably at least 100 times higher than the binding affinity to L-Quisqualic acid or S-(−)-5 fluorowillardine, and activates the NMDA receptor in a specific manner that can be blocked by one of the following NMDA antagonists: DL-AP5, DL-AP7, SDZ220-040, or Dexababidiol. Non-limiting examples of NMDA receptor agonists include NMDA, d-CYCLOSERINE, glycine, polyamines (such as spermidine), MILACEMIDE, homoquinolinic acid, and cis-ACPD. Non-limiting examples of AMPA receptor agonists include AMPA, polyamines, S-(−)-5-fluorowillardine, (RS)-Willardine, and Ampakines. Non-limiting examples of Kainate receptor agonists include kainic acid, Domoic acid, and SYM 2081. An increase in glutamate receptor activity may additionally be achieved by administering an agent which increases expression of a glutamate receptor in the injured brain.

Alternatively, the pharmaceutical composition administered in the methods of the present invention may increase the availability of glutamate receptor ligand for binding to the receptor. The pharmaceutical composition may, for example, increase a release of glutamate from cells in the injured brain. An increase in release of the neurotransmitter into the extracellular space would increase the levels of ligand available for receptor binding, thereby activating glutamate receptor activity. One of skill in the art will recognize that the identical result of increasing the release of glutamate into the extracellular space of the brain may be achieved with the administration of a compound that inhibits uptake of glutamate by cells in the injured brain. A compound that inhibits uptake of glutamate may inhibit the expression or activity of a glutamate transporter protein. Non-limiting examples of compounds that inhibit the uptake of glutamate by cells in the injured brain include 7-chlorokynurenic acid, Dihydrokainic acid, and SYM 2081. Alternatively, the pharmaceutical composition may comprise a compound selected from the group consisting of a positive modulator of glutamate receptor activity and a glutamate transport inhibitor, as are known in the art. Examples include Glycine, D-serine, and spermine.

One of skill in-the art will recognize that increasing glutamate receptor activity inherently causes activation of downstream signaling events. The NMDA receptor is a ligand-gated ion channel, and glutamate activation of the NMDA receptor results in channel opening and ion influx into the cell. Calcium influx through the NMDA receptor is known to activate the Ras/ERK pathway. Therefore it is an object of the present invention to include administration of a second messenger produced by activation of the glutamate-receptor associated signal transduction pathway, as well as their synthetic analogs and compounds which stimulate their production. Examples of second messengers produced by activation of the glutamate-receptor associated signal transduction pathway include NO and cGMP.

Administration of a pharmaceutical composition in the context of the present invention may be achieved locally to the brain or systemically, both of which are known in the art. Said composition may be administered alone or in combination with other therapies and may be administered in a physiologically acceptable carrier. One or more pharmaceutical compositions therapeutically effective to increase glutamate receptor activity in the brain may be administered to a single mammal simultaneously or at different times during the same treatment regimen.

Local administration of a pharmaceutical composition comprising a protein, nucleic acid, or peptide (or a cell comprising any of the same) with the ability to increase glutamate receptor activity in the brain may be achieved via gene therapy. Methods of delivering compositions to the brain are known in the art and may include the use of specialized catheters. Cells containing an expressible vector or vectors harboring the protein, nucleic acid, or peptide sequence may be induced to express the sequence to affect an increase in glutamate receptor activity. Although not required, such expressible sequences may include those of cloned NMDAR subunits. The present methods may alternatively be used in conjunction with stem cell replacement therapy whereby nerve cells damaged by glutamate receptor hyperactivation may be replaced with transplantable cells, and the transplantable cells, after being introduced into the injured brain, treated with a pharmaceutical composition therapeutically effective to increase glutamate receptor activity in the brain. Delivery of a pharmaceutical composition therapeutically effective to increase glutamate receptor activity would prevent any damage to the introduced cells, which may reside within the brain in an environment in which low levels of glutamate neurotransmitter are present. In this context, delivery of the pharmaceutical composition may be local or systemic.

The present invention also includes a combination therapy for treating a brain injury, whereby delivery of a pharmaceutical composition therapeutically effective to increase glutamate receptor activity in the brain of said mammal is preceded by delivery of a pharmaceutical composition therapeutically effective to decrease glutamate receptor activity. A method for treating a brain injury may thus comprise first administering to a mammal in need of such treatment a pharmaceutical composition comprising a glutamate receptor antagonist, wherein the composition is administered prior to or during an acute postinjury phase. The glutamate receptor antagonist may be an NMDA antagonist, and the NMDA antagonist may be selected from the group consisting of DL-AP5, DL-AP7, SDZ 220-040, and Dexanabinol. The term “acute postinjury phase” of said affliction is intended to include any time following injury, during which time functional NMDA receptor activity is increased to a level above normal. This method further comprises thereafter administering to the same mammal a pharmaceutical composition therapeutically effective to increase glutamate receptor activity in the brain of said mammal, wherein the pharmaceutical composition is only administered after the acute postinjury phase of said affliction. In this combination therapy, the pharmaceutical composition effective to decrease glutamate receptor activity and the pharmaceutical composition effective to increase glutamate receptor activity are administered exclusively of each other, meaning that they are not intended to be present in the mammal afflicted with the brain injury at the same time. The combination therapy provides the distinct advantage of maximizing the amount of time during which normal levels of glutamate receptor activity may be maintained. Because a pharmaceutical composition effective to increase glutamate receptor activity may only be administered after an acute postinjury phase, the combination therapy allows treatment of a brain injury to begin at an earlier stage of injury. Beginning treatment at an earlier stage of injury allows the damage that results from glutamate receptor hyperactivation in the earlier stages of the injury to be minimized. All aforementioned methods of the present invention relating to administration of a pharmaceutical composition therapeutically effective to increase glutamate receptor activity in the brain of said mammal may be used in conjunction with this combination therapy.

Finally, it is an object of the present invention to provide for a package comprising a first and a second pharmaceutical composition in dosage unit form, the first and second pharmaceutical compositions being suitable for use in the treatment of a brain injury. The first pharmaceutical composition in dosage unit form is therapeutically effective to decrease glutamate receptor activity in the brain of an injured mammal and may comprise a glutamate receptor antagonist. The glutamate receptor antagonist may be an NMDA antagonist, and the NMDA antagonist may be selected from the group consisting of DL-AP5, DL-AP7, SDZ 220-040, and Dexanabinol. The second pharmaceutical composition in dosage unit form is therapeutically effective to increase glutamate receptor activity in the brain of an injured mammal. Any of the aforementioned compounds described herein to increase glutamate receptor activity in the methods of the present invention may be included as the second pharmaceutical composition. The package also optionally contains instructions for administering the dosage unit form of the first pharmaceutical composition prior to or during an acute post injury phase of a brain injury and for subsequently administering the dosage unit form of the second pharmaceutical composition after the acute post injury phase of said affliction.

### Exemplification

### EXAMPLE I

### Dynamic Changes in N-methyl-D-aspartate Receptors after Closed Head Injury in Mice: Implications for Treatment of Neurological and Cognitive Deficits

Morphological Consequences of CHI. Closed head injury resulted in time-dependent pathological changes in brain morphology, including intraparenchymal bleeding and edema, as described (Chen et al., J. Neurotrauma 13, 557-568 (1996); Yatziv et al., J. Cereb. Blood Flow Metab. 22, 971-978 (2002); Beni-Adani et al., J. Pharmacol. Exp. Ther. 296, 57-63 (2001); Stahel et al., J. Cereb Blood Flood Metab. 20, 369-380 (2000); Shohami et al., J. Cereb. Blood Flood Metab. 23, 728-738 (2003); Grossman et al., NeuroImage 20, 1971-1981 (2003)). Animals killed 15 min to 24 h after CHI had some intracranial bleeding at the site of impact but no lesions. By day 7, all animals had a distinct cavitation lesion surrounded by dense gliosis corresponding to the area in the immediate vicinity of the impact. The brain on the side contralateral to the injury did not present bleeding or a lesion.

Dynamic Posttraumatic Changes in MK801 Binding to NMDAR. The density of activated NMDAR measured by quantitative autoradiography of MK801 in freshly frozen, unwashed brain sections (Porter & Greenamyre, Neurosci. Lett. 69, 105-108 (1994)) showed time-dependent changes in binding that varied with the brain region analyzed and the distance from the focal injury. Significant, bilateral increases in binding, presumably indicative of increased receptor activation and channel opening by increased efflux of endogenous glutamate, were measured 15 min postinjury in many regions posterior (in the “penumbra”) to the impact (Table 1). The largest increases (>50%) were seen in the hippocampus, especially the CA1 field and the dentate gyrus. The hippocampus contains the highest concentrations of NMDAR in the brain (Bowery et al., Br. J. Pharmacol. 93, 944-954 (1988)) and is intimately involved in memory function (Broersen, L. M., Prog. Brain Res. 126, 79-94 (2000); Brown & Aggleton, Nat. Rev. Neurosci. 2, 51-61 (2001); Baker & Kim, Learn. Mem. 9, 58-65 (2002)). Additional regions showing significant increases included the substantia innominata, amygdala, and several cortical and subcortical regions posterior and ventral to the impact (Table 1). In contrast, brain regions at close proximity to the impact showed a significant bilateral decrease in binding at this time point (Table 2). NMDAR open-channel binding in all regions declined progressively over time between 60 min and 8-24 h postinjury and remained low 1 week postinjury (Tables 1 and 2). The regions most affected by CHI, showing >50% reduction in binding, were the cortical regions closest to the impact (Table 2). However, significant decreases of 30-50% were also measured in the hippocampus, perirhinal cortex, and other regions posterior and ventral to the injury (Table 1). Seven days postinjury, binding was significantly lower on the injured (ipsilateral) compared to the contralateral side, in the brain regions closest to the impact (Table 2). These lateralized effects were not observed at earlier time points or in regions posterior and ventral to the impact (Table 1).

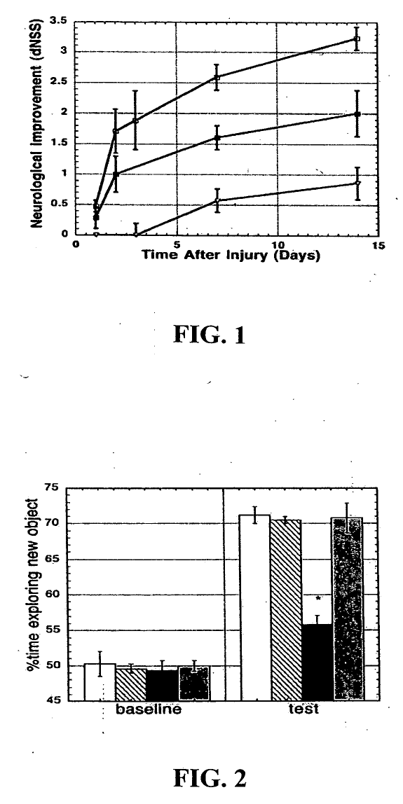

Effects of NMDA Treatment on Neurological and Cognitive Recovery After CHI. The above observations led us to speculate that starting as early as a few hours after CHI,:the injured animals are no longer likely to be in a hyperexcited state, because glutamate levels are reportedly no longer higher than normal (Benveniste et al., J. Neurochem. 43,1369-1374 (1984); Nilsson et al., J. Cereb. Blood Flow Metab. 10, 631-637 (1990); Schuhmann et al., J. Neurotrauma 20, 725-743 (2003); Bullock et al., Ann. N.Y. Acad. Sci. 765, 290-297 (1995); Davalos et al., Stroke 28, 708-710 (1997); Obrenovitch & Urenjak, J. Neurotrauma 14, 677-698 (1997); Obrenovitch et al.,, Int. J. Dev. Neurosci. 18, 281-287 (2000)), whereas NMDAR appear to be significantly hypo functional, possibly due at least in part to inhibition by an endogenous factor (Porter & Greenamyre, Neurosci. Lett. 69, 105-108 (1994); Quirion & Pert, NIDA Res. Monogr. 43, 217-223 (1983); McCoy & Richfield, Brain Res. 710, 103-111 (1996)). If such a blockade as well as frank loss of receptors were indeed contributing to the reduction in NMDAR binding and the neurological and cognitive deficits observed at these times, it could conceivably be overcome by increasing the levels of agonist stimulation through administration of a nontoxic dose of NMDA (Losada et al., Neuroendocrinology 57, 960-964 (1993); Von Lubitz et al., Eur. J. Pharmacol. 253, 95-99 (1994)). To test this hypothesis, we evaluated the effects of NMDA on motor and cognitive function after CHI. To achieve groups of animals with comparable trauma, the neurological severity score (NSS) was initially evaluated 1 h after CHI and animals randomized into three groups with similar mean initial NSS (6.14±0.14, 6.4±0.2, and 6.3±0.3), then assigned to receive vehicle, NMDA, or NMDA+MK801, respectively. NSS just before treatment initiation (24 h) was also similar in the three groups. Neurological recovery 7 and 14 days postinjury was significantly affected by NMDAR activation (χ² of 12.9 and 15.4, df of 2 and 2, and P=0.002 and P<0.0001 at 7 and 14 days postinjury, respectively). Comparison of the individual groups showed that recovery was significantly accelerated in the NMDA-treated mice when compared to saline 7 and 14 days postinjury (P=0.05 and 0.016, respectively; FIG. 1). Coadministration of the antagonist completely reversed the beneficial effect of the agonist (FIG. 1). Recovery in the NMDA+MK801 group was not only significantly worse than in the NMDA-alone group (P=0.001 and 0.0001 at 7 and 14 days, respectively), it was also significantly worse compared to the vehicle-treated mice (P=0.017 and 0.05 at 7 and 14 days, respectively; Mann-Whitney test; FIG. 1). NMDA-treated animals also performed significantly better than vehicle-treated controls in the object recognition test 14 days after CHI, at a dose which had no effect on performance in naive animals. All four groups of mice (naïve, naïve+NMDA, CHI, and CHI+NMDA) spent a similar proportion (≈50%) of time exploring two objects in an observation cage at baseline (FIG. 2). Four hours later, the mice were reintroduced into the cage in which one of the two “old” objects was replaced by a novel object. The vehicle-treated CHI mice spent a similar proportion of their time with the old and new object (FIG. 2), with no significant preference for the novel object, whereas the NMDA-treated injured mice significantly increased their exploration of the novel object compared to the familiar object (FIG. 2), to the same level (≈70%) as intact untreated mice and intact mice treated with NMDA.

TABLE 1
Effect of injury and time on regional distribution of NMDAR posterior to
the impact

| Region | Sham | 15 min | 1 h | 4 h | 8 h | 24 h | 7 days |
|---|---|---|---|---|---|---|---|
| Amygdala | | | | | | | |
| Left | 6.7 ± 0.6 | 8.67 ± 0.8 | 5.4 ± 0.6 | 4.8 ± 0.5* | 4.5 ± 0.7* | 5.5 ± 0.5 | 6.1 ± 0.4 |
| Right | 6.9 ± 0.6 | 9.0 ± 1.0† | 5.7 ± 0.4 | 5.2 ± 0.5 | 4.5 ± 0.7* | 5.7 ± 0.3 | 6.2 ± 0.6 |
| Anterior thalamus | | | | | | | |
| Left | 5.8 ± 0.37 | 6.8 ± 0.29 | 4.4 ± 0.45* | 3.5 ± 0.56* | 5.5 ± 0.32 | 4.9 ± 0.10* | 5.0 ± 0.46* |
| Right | 6.0 ± 0.39 | 6.5 ± 0.42 | 4.4 ± 0.34* | 3.5 ± 0.57* | 5.5 ± 0.32 | 4.7 ± 0.32* | 5.1 ± 0.48* |
| Dentate gyrus | | | | | | | |
| Left | 16.7 ± 3.2 | 27.2 ± 4.4† | 12.7 ± 1.3 | 10.2 ± 1.9 | 7.7 ± 1.1* | 12.7 ± 1.9 | 12.0 ± 1.1 |
| Right | 16.4 ± 3.5 | 26.3 ± 3.7† | 11.7 ± 2.2 | 10.4 ± 2.0 | 7.2 ± 1.2* | 12.1 ± 1.5 | 11.2 ± 0.8 |
| Dorsolateral striatum | | | | | | | |
| Left | 5.7 ± 0.34 | 6.7 ± 0.53† | 4.3 ± 0.33* | 3.9 ± 0.71* | 5.4 ± 0.38 | 4.6 ± 0.35* | 4.3 ± 0.42* |
| Right | 5.7 ± 0.45 | 7.0 ± 0.54† | 4.0 ± 0.45* | 3.8 ± 0.54* | 5.4 ± 0.39 | 4.4 ± 0.20* | 4.8 ± 0.43* |
| Hippocampus CA1 | | | | | | | |
| Left | 19.9 ± 4.0 | 33.0 ± 5.0† | 13.71 ± 1.3 | 13.8 ± 2.3 | 9.2 ± 1.2* | 14.4 ± 2.0 | 14.7 ± 1.4 |
| Right | 21.1 ± 4.9 | 36.5 ± 5.5† | 12.1 ± 2.4 | 13.2 ± 3.8 | 9.5 ± 2.0* | 14.4 ± 1.6 | 14.2 ± 1.2 |
| Hippocampus CA3 | | | | | | | |
| Left | 13.8 ± 2.1 | 19.0 ± 2.1† | 11.2 ± 1.0 | 8.7 ± 1.2* | 6.8 ± 1.1* | 10.4 ± 1.2 | 9.8 ± 0.6* |
| Right | 15.6 ± 3.1 | 20.2 ± 3.0 | 14.4 ± 4.4 | 10.7 ± 1.3 | 6.2 ± 1.0* | 10.4 ± 1.1 | 9.7 ± 0.5* |
| Hypothalamus | | | | | | | |
| Left | 3.8 ± 0.5 | 5.3 ± 0.4† | 3.4 ± 0.6 | 3.4 ± 0.4 | 2.8 ± 0.7 | 2.9 ± 0.2 | 3.4 ± 0.15 |
| Right | 4.0 ± 0.7 | 5.3 ± 0.27† | 3.14 ± 0.3 | 3.3 ± 0.2 | 3.1 ± 0.7 | 2.8 ± 0.1# | 3.4 ± 0.2 |
| Motor cortex | | | | | | | |
| Left | 9.7 ± 0.84 | 11.2 ± 1.3† | 5.8 ± 0.56* | 6.6 ± 1.0* | 6.9 ± 0.79* | 8.3 ± 0.56* | 5.3 ± 0.52* |
| Right | 9.7 ± 0.64 | 11.4 ± 1.1† | 6.0 ± 0.49* | 6.4 ± 1.0* | 6.7 ± 0.69* | 7.8 ± 0.45* | 6.2 ± 0.50* |
| Perirhinal cortex | | | | | | | |
| Left | 8.0 ± 0.6 | 9.16 ± 1.4 | 6.4 ± 0.3 | 5.1 ± 0.8* | 4.5 ± 1.0* | 5.4 ± 0.4* | 6.1 ± 0.4* |
| Right | 8.1 ± 0.85 | 9.0 ± 1.3 | 7.5 ± 0.3 | NA | 4.5 ± 1.0* | 5.6 ± 0.4* | 5.7 ± 0.4 |
| Piriform cortex | | | | | | | |
| Left | 7.9 ± 0.45 | 9.0 ± 1.3 | 5.8 ± 0.61* | 5.1 ± 0.83* | 6.0 ± 0.45* | 6.0 ± 0.23* | 5.3 ± 0.48* |
| Right | 7.9 ± 0.37 | 9.2 ± 0.75 | 5.9 ± 0.62* | 5.1 ± 0.66* | 6.2 ± 0.53* | 6.1 ± 0.36* | 5.7 ± 0.52* |
| Somatosensory cortex | | | | | | | |
| Left | 10.1 ± 0.64 | 11.7 ± 1.3† | 6.2 ± 0.56* | 6.8 ± 1.2* | 6.9 ± 0.72* | 8.2 ± 0.51* | 5.2 ± 0.54* |
| Right | 10.1 ± 0.71 | 12.2 ± 0.87† | 6.7 ± 0.63* | 6.8 ± 1.1* | 7.3 ± 0.86* | 8.4 ± 0.50* | 6.6 ± 0.53* |
| Substantia innominata | | | | | | | |
| Left | 3.7 ± 0.30 | 5.0 ± 0.32† | 3.5 ± 0.40 | 2.2 ± 0.36* | 4.5 ± 0.20* | 2.3 ± 0.31* | 3.1 ± 0.44 |
| Right | 3.5 ± 0.31 | 4.8 ± 0.22† | 3.6 ± 0.43 | 2.0 ± 0.32* | 4.5 ± 0.11* | 2.5 ± 0.27* | 3.1 ± 0.42 |
| Ventromedial striatum | | | | | | | |
| Left | 6.1 ± 0.27 | 6.9 ± 0.50† | 4.5 ± 0.40* | 4.1 ± 0.75* | 5.4 ± 0.37 | 4.9 ± 0.58* | 4.6 ± 0.43* |
| Right | 5.9 ± 0.41 | 7.0 ± 0.52† | 4.3 ± 0.42* | 4.1 ± 0.63* | 5.3 ± 0.35 | 4.6 ± 0.37* | 4.9 ± 0.46* |
| Ventral thalamus | | | | | | | |
| Left | 5.97 ± 0.5 | 6.2 ± 0.47 | 4.3 ± 0.4* | 4.6 ± 0.5* | 4.9 ± 0.3 | 4.3 ± 0.3* | 5.3 ± 0.35 |
| Right | 5.8 ± 0.7 | 6.3 ± 0.51 | 4.46 ± 0.5 | 4.3 ± 0.4* | 4.1 ± 0.5* | 4.3 ± 0.2* | 4.9 ± 0.4 |
| Brain sections from four to five animals per time point were collected at coronal levels posterior to the impact (levels ii and iii in Materials and Methods) and incubated with [3H]MK801 without washing or addition of glutamate and glycine. The table shows the mean ± SEM of density of specifically bound radioactivity in nCi/mg in regions in the uninjured (right) and injured (left) hemispheres. ANOVA (time) with repeated measures (region) | | | | | | | |
| # showed a significant effect of time and region on MK801 binding density. Individual regions where than tested by one-way ANOVA. Significance is defined as P < 0.05 by ANOVA followed by post-hoc Fisher's PLSD test. | | | | | | | |

*Lower than sham, P < 0.05.
†Higher than sham, P < 0.05.

TABLE 2
Effect of injury and time on regional distribution of NMDAR, coronal
level of impact

| Region | Sham | 15 min | 1 h | 4 h | 8 h | 24 h | 7 days |
|---|---|---|---|---|---|---|---|
| Corpus callosum | | | | | | | |
| Left | 4.8 ± 1.1 | 3.6 ± 0.8 | 3.0 ± 0.65 | 2.1 ± 0.22 | 2.6 ± 0.72 | 1.9 ± 0.26 | 3.7 ± 0.20 |
| Right | 5.0 ± 1.1 | 3.5 ± 0.9 | 3.0 ± 0.7 | 2.0 ± 0.28 | 2.5 ± 0.67 | 1.9 ± 0.19 | 3.9 ± 0.12 |
| Cingulate cortex | | | | | | | |
| Left | 14.1 ± 2.1 | 8.3 ± 2.2 | 7.3 ± 0.9 | 6.3 ± 1.2 | 4.7 ± 0.95 | 8.1 ± 0.90 | 5.9 ± 0.31 |
| Right | 14.4 ± 2.0 | 8.6 ± 2.4 | 7.2 ± 0.8 | 6.2 ± 1.1 | 4.6 ± 0.89 | 8.2 ± 0.93 | 6.6 ± 0.25 |
| Dorsolateral striatum | | | | | | | |
| Left | 8.7 ± 1.8 | 6.2 ± 1.6 | 4.9 ± 0.5 | 4.1 ± 0.56 | 4.0 ± 0.64 | 5.2 ± 0.66 | 5.0 ± 0.20 |
| Right | 8.9 ± 1.6 | 6.5 ± 1.7 | 4.8 ± 0.7 | 4.4 ± 0.71 | 4.2 ± 0.79 | 4.7 ± 0.52 | 5.3 ± 0.15 |
| Frontal motor cortex | | | | | | | |
| Left | 14.8 ± 2.2 | 8.5 ± 2.3 | 7.4 ± 0.83 | 6.6 ± 1.3 | 4.7 ± 0.83 | 8.2 ± 0.82 | 5.3 ± 0.24* |
| Right | 14.6 ± 2.2 | 8.6 ± 2.4 | 7.6 ± 0.86 | 6.3 ± 1.2 | 4.7 ± 0.82 | 8.0 ± 0.78 | 6.9 ± 0.36 |
| Somatosensory cortex | | | | | | | |
| Left | 15.1 ± 2.5 | 8.6 ± 2.4 | 7.1 ± 1.2 | 6.4 ± 1.5 | 4.8 ± 0.89 | 8.3 ± 0.80 | 5.4 ± 0.41* |
| Right | 14.8 ± 2.6 | 8.8 ± 2.6 | 7.4 ± 1.1 | 6.0 ± 1.1 | 5.0 ± 0.97 | 8.0 ± 0.83 | 7.0 ± 0.34 |
| Piriform cortex | | | | | | | |
| Left | 11.4 ± 2.0 | 6.7 ± 1.7 | 6.5 ± 0.88 | 4.7 ± 0.78 | 4.3 ± 0.76 | 5.6 ± 0.58 | 5.6 ± 0.23 |
| Right | 11.1 ± 2.2 | 7.1 ± 1.9 | 6.3 ± 0.84 | 4.4 ± 0.55 | 4.5 ± 0.76 | 5.7 ± 0.68 | 5.9 ± 0.25 |
| Ventromedial striatum | | | | | | | |
| Left | 9.3 ± 1.7 | 6.6 ± 1.7 | 5.0 ± 0.70 | 4.5 ± 0.61 | 4.2 ± 0.81 | 5.3 ± 0.63 | 5.2 ± 0.26 |
| Right | 9.4 ± 1.6 | 6.6 ± 1.7 | 5.0 ± 0.84 | 4.4 ± 0.80 | 4.3 ± 0.78 | 5.1 ± 0.54 | 5.5 ± 0.25 |
| Brain sections were collected from the level of impact (level i in Materials and Methods) and treated as described. Data show the mean ± SEM of density of specifically bound radioactivity in nCi/mg in regions in the uninjured (right) and injured (left) hemispheres. ANOVAwith repeated measures showed a significant reduction (P < 0.05) in NMDA receptor density of all regions at all time points when compared to sham, with the exception | | | | | | | |
| # of the corpus callosum at 7 days (P 0.088). Significance is defined as P < 0.05 by ANOVA followed by post hoc PLSD test | | | | | | | |

*Left (injured) lower than right, P < 0.05.

Materials and Methods

Trauma Model. We used 8- to 12-week-old male Sabra mice (30-35 g) kept under controlled temperature and light conditions with food and water available ad libitum. The study was approved by the Institutional Animal Care Committee of the Hebrew University. Experimental CHI was induced by using a modified weight-drop device developed in our laboratory (Chen et al., J. Neurotrauma 13, 557-568 (1996); Yatziv et al., J. Cereb. Blood Flow Metab. 22, 971-978 (2002)). Briefly, after induction of ether anesthesia, a midline longitudinal incision was performed, the skin was retracted and the skull was exposed. The left anterior frontal area was identified and a Teflon tipped cone (2-mm diameter) was placed 1 mm lateral to the midline, in the midcoronal plane. The head was held in place manually and a 75-g weight was dropped on the cone from a height of 18 cm, resulting in a focal injury to the left hemisphere. After trauma, the mice received supporting oxygenation with 95% O₂ for no longer than 2 min and were then returned to their cages. Sham controls received anesthesia and skin incision only. For autoradiographic studies, groups of four to five animals per time point were killed 15 min, 1, 4, 8, or 24 h, or 7 days postinjury and brains were quickly removed and frozen on dry ice.

Autoradiography of Activated (Open Channel State) NMDAR. Autoradiography for activated NMDAR distribution was performed on freshly frozen, unwashed brain.-sections as described by Porter and Greenamyre (Porter & Greenamyre, Neurosci. Lett. 69, 105-108 (1994)) with small modifications. Six parallel series of consecutive cryostat sections (10 μm, cut at −15° C. and thawmounted onto coated glass slides) were produced in the coronal plane and collected at 100- to 200-μm intervals from the prefrontal cortex to the cerebellum. The preincubation stage (meant to facilitate removal of endogenous glutamate and other water-soluble coactivators or blockers) was intentionally omitted (Bowery et al., Br. J. Pharmacol. 93, 944-954 (1988); Porter & Greenamyre, Neurosci. Lett. 69, 105-108 (1994)). Sections were incubated directly with a very small volume (10-100 μl, depending on the size of the section) of 10 nM [³H]MK801 in 50 mM Tris-acetate buffer at pH 7.4 for 4 h at room temperature, without addition of exogenous glutamate and glycine to the incubation mixture. Binding density under these conditions is proportional to the actual density of activated (open channel) NMDAR in the individual brain and region in situ. Nonspecific binding was measured on a second series by incubating the radioactive ligand in the presence of excess (5 μM) unlabeled MK801, and sections were washed, dried, and apposed to film with calibrated tritium micro scales (Amersham Pharmacia) as described (Porter & Greenamyre, Neurosci. Lett. 69, 105108 (1994); Biegon et al., J. Neurochem. 82, 924-934 (2002)). The resulting autoradiograms of sections and standards were digitized simultaneously by using a large-bed UMAX scanner. A third series was stained with cresyl violet for anatomical verification of the lesion site and regions of interest. Sections from the remaining three series were labeled with iodoMK801 (Gibson et al., Int. J. Rad. Appl. Instrum. B 19, 319-326 (1992)) and used for experiments involving manipulations of incubation conditions (data not shown).

Image Analysis. Scanned images were analyzed quantitatively (Rainbow et al., J. Neurosci. Methods 5, 127-138 (1982); Biegon, A., in Brain Imaging: Techniques and Applications (Ellis Horwood, Chichester, U.K.), pp. 130-143 (1989)) by using NIH IMAGE software. Anatomical regions underlying the autoradiographic images were identified on the consecutive brain sections stained with cresyl violet in reference to a mouse brain atlas (Paxinos & Franklin, The Mouse Brain in Stereotaxic Coordinates (Academic, San Diego) (2001)). Regions of interest were grouped in three levels by distance from the impact, as follows: level 1 (anterior striatal level, Bregma 1.7 to 0.02) contained sections in the direct path of the impact; level 2 (posterior striatal level, Bregma −0.22 to −0.82) contained regions directly posterior to the impact; and level 3 (dorsal hippocampal level, Bregma −1.2 to −2.06) contained regions more remote (posterior) from the site of impact. Gray levels of seven to eight regions per anatomical level were measured in triplicates on the left and right separately; the gray levels were converted to nCi/mg (1 Ci=37 GBq) via the calibrated standard curve. Nonspecific binding was similarly measured for each region and animal, and the values of nonspecific binding subtracted from total binding to yield specific binding. The mean specific binding values in the various regions of sham treated animals were then compared statistically to the mean values in CHI animals killed at various times after the injury.

Drug Treatments. The NMDAR agonist NMDA and the noncompetitive antagonist MK801 were dissolved in saline and injected i.p. in groups of eight to nine animals per treatment. Three groups of animals were used to test the effect of NMDAR activation on neurological recovery: (i) head injury followed by saline vehicle 1 and 2 days later (controls); (ii) head injury followed by NMDA 20 mg/kg, 1 and 2 days later; or (iii). head injury followed by NMDA 20 mg/kg combined with MK801 1 mg/kg, both administered 1 and 2 days after the injury. The three groups were repeatedly evaluated for neurobehavioral deficits over a 2-week period (see below). The NMDA dose was chosen from a dose-response experiment in intact mice in which we tested doses between 20 and 100 mg/kg. The lowest dose produced hyperactivity and some stereotypy but no convulsions or mortality, whereas significant mortality was observed at doses above 60 mg/kg. Cognitive function was evaluated in head-injured and intact rats administered with either NMDA 20 mg/kg or saline 24 and again 48 h postinjury and tested 14 days later for performance in the object recognition test (see below).

Neurobehavioral Evaluation. The neurological severity score (NSS) is a 10-point scale that assesses the functional neurological status of mice based on the presence of various reflexes and the ability to perform motor and behavioral tasks such as beam walking, beam balance, and spontaneous locomotion (Beni-Adani et al., J. Pharmacol. Exp. Ther. 296, 5763 (2001)). Animals are awarded one point for failure to perform one item, such that scores can range from zero (healthy uninjured animals) to a maximum of 10, indicating severe neurological dysfunction, with failure at all tasks. The NSS obtained 1 h after trauma reflects the initial severity of injury and is inversely correlated with neurological outcome. Animals were evaluated 1 h after CHI, and 1, 2 or 3, 7, and 14 days later. Mice were randomized to the three groups after the initial evaluation to ensure similar initial severity values. Each animal was assessed by an observer who was blinded to treatment. The extent of recovery (dNSS) was calculated as the difference between the NSS at 1 h and at any subsequent time point. Thus, a positive dNSS reflects recovery, a 0 reflects no change, and a negative dNSS reflects neurological deterioration.

Evaluation of Performance in the Object Recognition Test. The object recognition test was performed as originally described by Ennaceur and Delacour (Ennaceur & Delacour, Behav. Brain Res. 31, 47-59 (1988)). In the first part of the test (14 days after CHI) mice were placed in the testing cage (a glass aquarium-like transparent box of 60×60 cm) for 1 h habituation. On the following day they were put back into the same cage with two identical objects. The cumulative time spent by the mouse at each of the objects was recorded manually during a 5-min interval by an observer blinded to the treatment received. Four hours later, the mice were reintroduced into the cage, where one of the two objects was replaced by a new one. The time (of 5 min total) spent at each of the objects was recorded. The basic measure is the percent of the total time spent by mice in exploring an object during the testing period, whereby normal healthy rodents will spend relatively more time exploring a new object than a familiar, i.e., “memorized” object.

1. A method for treating a brain injury, the method comprising administering to a mammal afflicted with a brain injury a pharmaceutical composition therapeutically effective to increase glutamate receptor activity in the brain of said mammal, wherein the pharmaceutical composition is only administered after an acute postinjury phase of said affliction.

2. The method of claim 1 wherein the mammal is a human.

3. The method of claim 2 wherein the pharmaceutical composition is administered at a timepoint of at least about 6 hours postinjury.

4. The method of claim 1 wherein the pharmaceutical composition is administered intermittently or continuously resulting in a duration of at least 48 hours treatment.

5. The method of claim 1 wherein the pharmaceutical composition comprises a glutamate receptor agonist.

6. The method of claim 5 wherein the glutamate receptor agonist is selected from the group consisting of an indirect glutamate receptor agonist, a direct glutamate receptor agonist, and a partial glutamate receptor agonist.

7. The method of claim 5 wherein the glutamate receptor agonist is selected from the group consisting of an NMDA receptor agonist, a Kainate receptor agonist, and an AMPA receptor agonist.

8. The method of claim 1 wherein the pharmaceutical composition administered increases a release of glutamate from cells in the injured brain.

9. The method of claim 1 wherein the pharmaceutical composition administered inhibits an uptake of glutamate by cells in the injured brain.

10. The method of claim 1 wherein the pharmaceutical composition administered increases expression of a glutamate receptor in the injured brain.

11. The method of claim 1 wherein the pharmaceutical composition comprises a compound selected from the group consisting of a positive modulator of glutamate receptor activity and a glutamate transport inhibitor.

12. The method of claim 1 wherein the pharmaceutical composition is administered locally to the brain or systemically.

13. The method of claim 1 wherein the brain injury is caused by an event selected from the group consisting of trauma, ischemia, irradiation, meningitis, surgery, and encephalitis.

14. The method of claim 13 wherein the brain injury is caused by ischemia, and the ischemia is caused by a stroke.

15. A method for treating a brain injury, the method comprising:
a. administering to a mammal in need of such treatment a pharmaceutical composition comprising a glutamate receptor antagonist, wherein the composition is administered prior to or during an acute postinjury phase; and
b. thereafter administering to the mammal of step a) a pharmaceutical composition therapeutically effective to increase glutamate receptor activity in the brain of said mammal, wherein the pharmaceutical composition is only administered after-the acute postinjury phase of said affliction.

16. The method of claim 15 wherein the mammal is a human.

17. The method of claim 16 wherein the pharmaceutical composition of step b) is administered at a timepoint of at least about 6 hours postinjury.

18. The method of claim 15 wherein the pharmaceutical composition of step b) is administered intermittently or continuously resulting in a duration of at least 48 hours treatment.

19. The method of claim 15 wherein the pharmaceutical composition of step b) comprises a glutamate receptor agonist.

20. The method of claim 19 wherein the glutamate receptor agonist is selected from the group consisting of an indirect glutamate receptor agonist, a direct glutamate receptor agonist, and a partial glutamate receptor agonist.

21. The method of claim 19 wherein the glutamate receptor agonist is selected from the group consisting of an NMDA receptor agonist, a Kainate receptor agonist, and an AMPA receptor agonist.

22. The method of claim 15 wherein the pharmaceutical composition administered in step b) increases a release of glutamate from cells in the injured brain.

23. The method of claim 15 wherein the pharmaceutical composition administered in step b) inhibits an uptake of glutamate by cells in the injured brain.

24. The method of claim 15 wherein the pharmaceutical composition administered in step b) increases expression of a glutamate receptor in the injured brain.

25. The method of claim 15 wherein the pharmaceutical composition of step b) comprises a compound selected from the group consisting of a positive modulator of glutamate receptor activity and a glutamate transport inhibitor.

26. The method of claim 15 wherein the pharmaceutical composition of step b) is administered locally to the brain or systemically.

27. The method of claim 15 wherein the brain injury is caused by an event selected from the group consisting of trauma, ischemia, irradiation, meningitis, surgery, and encephalitis.

28. The method of claim 27 wherein the brain injury is caused by ischemia, and the ischemia is caused by a stroke.

29. A package of two separate pharmaceutical compositions in dosage unit form comprising:
a. a first pharmaceutical composition in dosage unit form comprising a glutamate receptor antagonist;
b. a second pharmaceutical composition in dosage unit form therapeutically effective to increase glutamate receptor activity; and optionally c. instructions for administering the dosage unit form of (a) prior to or during an acute post injury phase of a brain injury and for subsequently administering the dosage unit form of (b) after the acute post injury phase of brain injury.

30. The method of claims 7 or 21 wherein the glutamate receptor agonist is an NMDA receptor agonist, and the NMDA receptor agonist is selected from the group consisting of NMDA, d-CYCLOSERINE, glycine, polyamines, MILACEMIDE, homoquinolinic acid, and cis-ACPD.

31. The method of claims 7 or 21 wherein the glutamate receptor agonist is an AMPA receptor agonist, and the AMPA receptor agonist is selected from the group consisting of AMPA, polyamines, S-(−)-5-fluorowillardine, (RS)-Willardine, and Ampakines.

32. The method of claims 7 or 21 wherein the glutamate receptor agonist is a Kainate receptor agonist, and the Kainate receptor agonist is selected from the group consisting of kainic acid, Domoic acid, and SYM 2081.

33. The method of claim 15 wherein the glutamate receptor antagonist is an NMDA antagonist.

34. The method of claim 33 wherein the NMDA antagonist is selected from the group consisting of DL-AP5, DL-AP7, SDZ 220-040, and Dexanabinol.

35. The method of claims 9 or 23 wherein the pharmaceutical composition which inhibits an uptake of glutamate by cells in the injured brain comprises a member selected from the group consisting of 7-chlorokynurenic acid, Dihydrokainic acid, and SYM 2081.
